(19) United States
(12) Patent Application Publication
(10) Pub. No.: US 2008/0019476 A1
(43) Pub. Date: Jan. 24, 2008

(54) Apparatus and Method for Producing Medical X-ray Images

(75) Inventor: Armen Mirzayan, Glendale, CA (US)

Correspondence Address: FITCH EVEN TABIN AND FLANNERY, 120 SOUTH LA SALLE STREET, SUITE 1600, CHICAGO, IL, 60603-3406, US

(73) Assignee: ARMEN MIRZAYAN, DDS, INC., Los Angeles, CA (US)

(21) Appl. No.: 11/459,620

(22) Filed: Jul. 24, 2006

(51) Int. Cl. A61B 6/14; G01T 1/20; G01T 1/24

(52) U.S. Cl. 378/38; 250/370.11

(57) ABSTRACT

An x-ray image sensor has an array disposed between an x-ray source and a photo-responsive device, such as a CCD or CMOS device. The array is comprised of a plurality of generally linearly-shaped, opaque elements that are spaced apart from each other by a predetermined distance and are adapted to absorb either x-ray radiation or light radiation. When a plurality of x-ray images is taken of the same object from different angles, images of the array are superimposed on the images of the object. Using a CAD program or other algorithm, the differences in spacing between the array elements for each image are used to derive a three-dimensional image of the object.

### FIELD OF INVENTION

This relates to a medical x-ray imaging method and apparatus. More particularly, this relates to a method and apparatus for producing three-dimensional medical x-ray images.

### BACKGROUND

The medical and dental fields have numerous methods for screening and diagnosing various debilitating conditions. Laboratory tests and x-ray examinations have been an integral part of these examinations for many years, but newer technologies like MRI's often provide greater accuracy and information, although at a higher cost.

One limitation of x-ray imaging is that this provides two-dimensional pictures or images of three-dimensional objects. This sometimes generates errors by masking critical data because of the superimposition of objects upon one another that differ in opacity. Frequently, practitioners must take x-rays from differing angles to be able to ascertain greater detail, but this often leaves too much to interpretation by the practicing clinician.

Traditionally, x-ray radiography capitalizes on the difference in opacity (level of whiteness) and lucency (level of darkness). Mineralized hard tissue, such as bone, tooth enamel and dentin, shows up as opaque, whereas soft tissue, like muscle or gum tissue, does not appear on x-ray film. This discrepancy allows clinicians to differentiate anatomical variations and render a diagnosis. For instance, an infection of a tooth spreads to the apex (bottom) and then to the jaw bone. The infection then “eats” away at the bone and demineralizes (removes the minerals) from the bone. This converts the initial opaque status of the bone to a lucent one, allowing a dentist to determine that there is an infection present. Indeed, that is precisely how a cavity in the tooth is diagnosed; the bacteria progressively remove minerals from tooth structure until they become a certain size and are readily apparent as a radiolucent lesion on an x-ray film.

The amount and density of the minerals, namely calcium/hydroxyappetite, allow the differentiation of anatomical structures. The amount of minerals in enamel outweighs the amount found in bone, dentin, or cementum. Hence, enamel on tooth structure appears more opaque than the latter anatomical structures, allowing for proper identification and delineation. The same is true of bone, ligaments, and tendons. X-rays can reveal ligaments, which are also mineralized, but not as densely as bone, whereas tendons are not. Therefore, it is easy to differentiate those structures from each other.

Currently, in the field of dentistry most x-ray readings are taken by one of three distinct methods: traditional x-ray film, a charge coupled device (CCD) sensor, a complementary metal oxide semiconductor (CMOS) device sensor, or a phosphorous plate. Traditional films require the exposure of an object onto a film, with an x-ray beam. This film is then processed with a developer solution and a fixer solution. The process usually takes well over 5 minutes to render an acceptable image for viewing on a lighted view box.

With the advent of CCD and CMOS technology, x-rays can now be received by a digital intraoral sensor that is read by a capture card in a computer. The sensor replaces the traditional film in the mouth. The image is displayed on a monitor for viewing in just seconds. This eliminates the time it takes for processing the images.

The digitalization of x-ray images has had a profound impact. Once digitized, various calculations and algorithms can be applied to ascertain pertinent information. X-ray images can be digitized in different ways. A digital image of the x-ray film (that was conventionally developed) can be taken with a digital camera or scanned by an optical scanner which will produce a data file of the image in a TIFF, RAW, JPG, BMP, GIFF or other data format. A digital sensor as described above can be used to capture the digital image format as well.

X-rays have been useful in identifying cancer, trauma, and even tooth decay for many years. However, their main limitation is that they rely on the contrast of opacity and lucency of objects to render an image. Another limitation is that they render a two-dimensional image. Furthermore, they allow for more opaque structures to mask more lucent structures, ultimately leading to the need for more expensive technology to overcome those shortcomings. Thus there is a need for improved, yet relatively inexpensive, methods and devices for overcoming some of these shortcomings.

### SUMMARY OF THE ILLUSTRATED EMBODIMENTS

Embodiments of the invention include an x-ray image sensor having an array disposed between an x-ray source and a photo-responsive device, such as a CCD or CMOS device. The array is comprised of a plurality of generally linearly-shaped, opaque elements that are spaced apart from each other by a predetermined distance and are adapted to absorb either x-ray radiation or light radiation. When a plurality of x-ray images is taken of the same object from different angles, images of the array are superimposed on the images of the object. Using a CAD program or other algorithm, the differences in spacing between the array elements for each image are used to derive a three-dimensional image of the object.

In one aspect, an x-ray image sensor comprises a housing, a scintillator disposed in the housing and adapted to convert the x-rays to light signals, and a photo-responsive device disposed in the housing and adapted to convert the light signals to electric signals. The photo-responsive device can be a CCD or a CMOS device. The sensor further includes an array disposed between an x-ray source and the photo-responsive device when x-rays are being received. The array includes a plurality of generally linearly-shaped opaque elements. Each of the elements is adapted to absorb x-ray radiation or light radiation, and are spaced apart from one another by a predetermined distance.

In one aspect, the housing includes a housing wall having an outer surface and an inner surface. The array is disposed either on the outer surface, on the inner surface or between the outer and inner surfaces of the housing wall.

In an alternative embodiment, a sensor comprises a housing and a scintillator disposed in the housing and adapted to convert a first portion of the x-rays to light signals. A photo-responsive device is disposed in the housing and adapted to convert a first portion of the light signals to electric signals. The photo-responsive device includes a first array adapted to prevent conversion of a second portion of the light signals to other electric signals. The first array includes a plurality of generally linearly-shaped elements disposed in a generally parallel, spaced-apart relationship from one another by a predetermined distance.

In one aspect, the photo-responsive device is a CCD having a capacitor array. The plurality of generally linearly-shaped elements is comprised of a plurality of disabled capacitors or a plurality of omitted capacitors disposed within the capacitor array.

In another aspect, the photo-responsive device is a CMOS device having a pixel array. The plurality of generally linearly-shaped elements is comprised of a plurality of disabled pixels or a plurality of omitted pixels disposed within the pixel array.

In another aspect, the photo-responsive device is a CCD having a capacitor array. The first array is comprised of a mask disposed on the capacitor array. Each of the plurality of generally linearly-shaped elements is adapted to absorb light radiation.

In yet another aspect, the photo-responsive device is a CMOS device having a pixel array. The first array is comprised of a mask disposed on the pixel array. Each of the plurality of generally linearly-shaped elements is adapted to absorb light radiation.

There are additional aspects to the present inventions. It should therefore be understood that the preceding is merely a brief summary of some embodiments and aspects of the present inventions. Additional embodiments and aspects are referenced below. It should further be understood that numerous changes to the disclosed embodiments can be made without departing from the spirit or scope of the inventions. The preceding summary therefore is not meant to limit the scope of the inventions. Rather, the scope of the inventions is to be determined by appended claims and their equivalents.

### BRIEF DESCRIPTION OF THE DRAWINGS

These and/or other aspects and advantages of the present invention will become apparent and more readily appreciated from the following description of certain embodiments, taken in conjunction with the accompanying drawings of which:

FIG. 1 is a simplified diagram of an apparatus for providing a three-dimensional, dental x-ray image in accordance with an embodiment of the invention;

FIG. 2 is a simplified cross-section diagram of the sensor of FIG. 1.;

FIG. 3 is a simplified perspective view of a portion of a housing wall inner surface of the sensor of FIG. 1;

FIGS. 4a, 4b and 4c are simplified illustrations of two-dimensional x-ray images of a tooth generated in accordance with an embodiment of the invention; and

FIG. 5 is a flow diagram showing a method of obtaining a three-dimensional image of an object according to an embodiment of the invention.

### DETAILED DESCRIPTION

The following description is of the best mode presently contemplated for carrying out the invention. Reference will be made in detail to embodiments of the present invention, examples of which are illustrated in the accompanying drawings, wherein like reference numerals refer to like elements throughout. It is understood that other embodiments may be used and structural and operational changes may be made without departing from the scope of the present invention.

Embodiments of the invention are intended to remedy certain shortcomings inherent in two-dimensional x-ray images by using algorithms to calculate images of anatomical structures with the use of Computer Aided Design (CAD) programs and render a three-dimensional view of the structure that can be viewed from differing angles. Known methods of CAD render a three-dimensional model from a two-dimensional object, but they use some contrasting medium or different established templates to ascertain the working model.

For example, Rivera, et al, U.S. Pat. No. 6,341,153, discloses the use of CAD/CAM software in a non-destructive manner by examining three-dimensional CT imagery. This is accomplished by comparing the CT image of an object to preloaded CT images of stored drawings of parts in another database.

Vaillant, U.S. Pat. No. 6,404,843, discloses a method of reconstruction of a three-dimensional image of sharp contrast from a set of two-dimensional images of an object comprising the elements of sharp contrast. Then, for each different position of an x-ray camera around the object, a two-dimensional image is taken, and the use of an algorithm for reconstruction of the 3D image is preceded by a stage of filtering of the set of two-dimensional images derived from the contrast.

Embodiments of the present invention differ by using a meshwork of fiber elements, which serves as a grid or array for anatomical landmark and reference. The fiber elements are disposed on or within a sensor housing or at other locations between an x-ray source and a photo-responsive device, such as a CCD or CMOS device. There are no pre-existing templates or contrasting medium from which to derive a working model; the information comes from the object itself when its image is captured on the array.

In one embodiment, an array is comprised of a plurality of elements, each of which is constructed of a strand of fiber that is impregnated with an opaque medium, such as for example, barium. This medium is in stark contrast to any anatomical structure, and each fiber element is spaced apart from each adjacent fiber element by a predetermined distance. This serves as a meshwork or a grid that appears on the x-ray images, thus providing the computer with an orientation and location of an object, in all spatial fields. This allows the CAD or other software to compute the appropriate dimensions of the working model. Furthermore, the opaque medium that is embedded into the meshwork has associated color values (hue, value, and chroma) that are distinctly different than any hard tissue found in human anatomy. This allows for the elimination of the grid when the final images are analyzed, in order to eliminate any artifact from the true working three-dimensional model.

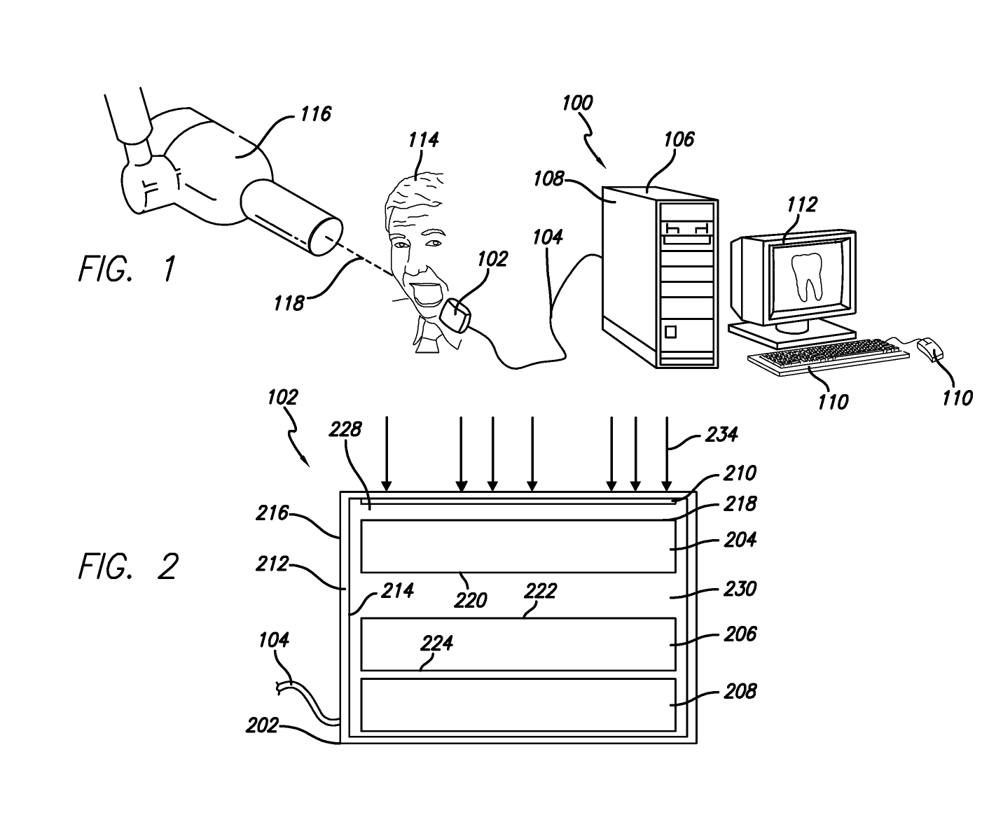

FIG. 1 shows an apparatus 100 for providing a three-dimensional dental x-ray image. An intraoral sensor 102 adapted to produce electrical signals corresponding to dental x-ray images is connected via a cable 104 to a computer system 106. Alternatively the sensor 102 could communicate with the computer system 106 via a wireless communication link. The computer system 106 includes a housing 108 that encloses a processor, a system memory, preferably including both high speed random access memory (RAM) and non-volatile memory, such as read only memory (ROM), erasable or alterable non-volatile memory (e.g., flash memory), and a mass storage device, such as a hard disk drive, for storing operating system programs, data, application programs, etc. For simplicity of illustration, these components located within the housing 108 are not shown in FIG. 1.

User input devices 110 include a keyboard and mouse for entering user commands into the computer system 106. A display 112 provides visual computer system output for the user.

The sensor 102 can be placed in the mouth of a person 114 and positioned on one side of a tooth (not shown) for which an image is desired. This permits an x-ray source 116 to be placed on the other side of the tooth and aligned in a first position so that x-rays 118 streaming from the x-ray source 116 can pass into the person's mouth, through the tooth and onto the sensor 102. The x-rays will strike the sensor 102 at a first average angle of incidence to the sensor 102 thus causing it to produce a first set of electrical signals corresponding to a two-dimensional image of the tooth. This first set of electrical signals can be sent to the computer system 106 for conversion to and storage of a set of data that corresponds to these signals.

The x-ray source 116 can be moved into a second position relative to the tooth and the sensor 102 so that the x-rays 118 are still directed toward the same tooth and the sensor 102. The sensor 102 remains in a generally unchanged position or orientation with respect to the tooth. Thus x-rays will travel from the x-ray source 116, pass through the mouth and the tooth, and strike the sensor 102 at a second average angle of incidence to the sensor 102. This allows the sensor 102 to generate a second set of electrical signals corresponding to another two-dimensional image of the tooth taken from a different perspective. The second set of electrical signals can then be sent to the computer system 106 for conversion to and storage of a set of data corresponding to the second set of electrical signals. As will be explained in more detail below, the processor of the computer system 106 is adapted to execute a program that converts the data thus far received into another set of data that corresponds to a three-dimensional image of the tooth. This three-dimensional image can be presented on the display 112 for analysis and medical diagnosis.

FIG. 2 is a simplified cross-section diagram of the sensor 102 of FIG. 1. The sensor 102 includes a housing 202, a scintillator 204, an optic device 206, a photo-responsive device 208, and an array 210. The housing 202 has a wall 212 comprised of an inner surface 214 and an outer surface 216, and is constructed of plastic or any other material that is generally transparent to x-ray radiation. The scintillator 204 is disposed in the housing 202 and adapted convert x-rays 234 into light signals. The scintillator 204 has a proximate surface 218 that receives the x-rays 234 and a distal surface 220 that emits the light signals as the scintillator performs its conversion function. The proximate surface 218 is in a spaced-apart relationship from the housing wall 212 and the array 210, and thus a first cavity 228 is defined by the wall 212 and the array 210, on the one hand, and the proximate surface 218 of the scintillator 204, on the other hand.

The photo-responsive device 208 is disposed in the housing 202 facing the distal surface 220 of the scintillator 204. The photo-responsive device 208 converts the light signals generated by the scintillator 204 into electrical signals that are transferred via the cable 104 to the computer system 106 (FIG. 1). The photo-responsive device 208 can be a CCD, a CMOS device, or any other device that performs a similar function.

The optic device 206 is disposed within the housing 202 between the scintillator 204 and the photo-responsive device 208 and is in a spaced-apart relationship with the scintillator 204. Thus a second cavity 230 is defined by the housing 202, the scintillator 204 and the optic device 206. The optic device 206 channels the light signals emitted from the scintillator 204 onto the photo-responsive device 208. The optic device 206 has a proximate side 222 adapted to receive the light signals from the scintillator 204 and a distal side 224 that abuts the photo responsive device 208 and emits the channeled light signals to the photo-responsive device 208. According to one embodiment, the optic device 206 is a fiber optic plate comprising a plurality of glass fibers bundled together and each having a very small diameter. In addition to channeling the light signals, the fiber optic plate has x-ray absorption characteristics, so that only a small percentage of the x-rays enter the CCD, resulting in a less noisy image.

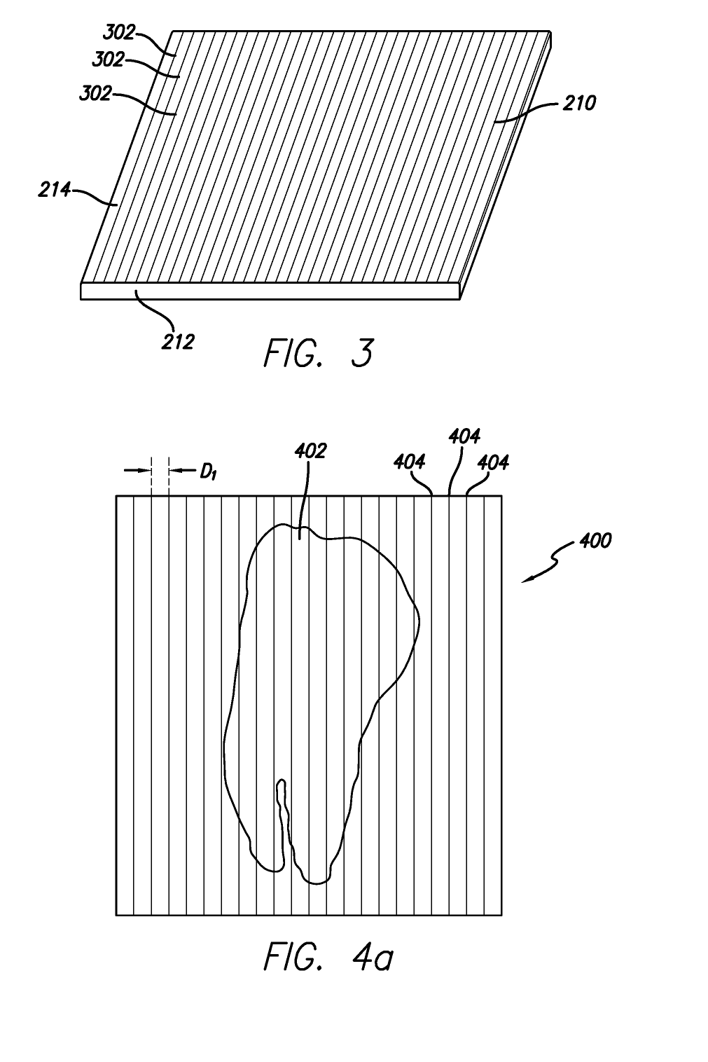

The array 210 is disposed on the inner surface 214 of the housing wall 212 adjacent to the proximate surface 218 of the scintillator 204. FIG. 3 is a perspective view of a portion of the housing wall inner surface 214 with the array 210 disposed thereon. The array 210 is comprised of a plurality of generally linearly-shaped opaque elements 302, each of which is adapted to absorb x-rays. Each of the elements 302 is spaced apart from one another by a predetermined distance and have a predetermined thickness. The elements 302 forming the array 210 are constructed of a barium impregnated fiber. However alternative embodiments include any other material that absorbs x-rays. While the elements 302 of FIG. 3 are parallel, generally straight lines that are spaced apart equally from one another, other embodiments can include elements forming other geometries as well, including generally linearly-shaped elements forming an arcuate pattern, a sinuous pattern, a checkerboard pattern, etc., wherein at least a portion of the elements are in a parallel, spaced-apart relation of a predetermined distance from one another.

While the embodiment of FIG. 2 discloses an array that is disposed on the inner surface 214 of the housing wall 212, other embodiments of the invention include different locations of the array 210. Generally, an array can be placed in any one of various positions between the x-ray source and the photo-responsive device 208. For example, other locations for the array include:
(a) on the housing wall outer surface 216;
(b) embedded in the housing wall 212 between the housing wall outer and inner surfaces 216, 214;
(c) in the first cavity 228 between the housing wall 212 and the array 210, on the one hand, and the scintillator proximate surface 218, on the other hand;
(d) on the scintillator proximate surface 218;
(e) on the scintillator distal surface 220;
(f) in the second cavity 230 between the scintillator distal surface 220 and the optic device 206 (or the cavity between the scintillator 204 and the photo-responsive device 208 for sensors lacking the optical device 206);
(g) on the optic device proximate side 222; and
(h) on the optic device distal side 224.
   Depending upon the location, the array 210 is constructed of a material that absorbs x-rays for some locations or light for other locations. For embodiments having arrays disposed in locations (a)-(d) above, the arrays are constructed of a material that absorbs x-rays. For the locations (e)-(h) above, the arrays are constructed of a material that absorbs light.

In addition to the various locations for the array listed above, other embodiments of the invention include arrays or array-equivalents that are part of a photo-responsive device. For example according to one embodiment, a photo-responsive device includes a first array (or array-equivalent) that is adapted to prevent conversion of a portion of the received light signals into electric signals. In the case of CCD or CMOS devices, this is accomplished by disabling or omitting a portion of the capacitors in the capacitor array or a portion of the pixels in the pixel array of these devices. Those disabled or omitted capacitors or pixels form a pattern or array that effectively is comprised of a plurality of generally linearly-shaped elements disposed in a generally parallel, spaced-apart relationship from one another by a predetermined distance.

In yet another exemplary embodiment, an array is comprised of a mask disposed on the capacitor array or pixel array of a photo-responsive device. The mask array is comprised of a plurality of generally linearly-shaped elements that are adapted to absorb light radiation and that are disposed in a generally parallel, spaced-apart relationship from one another by a predetermined distance.

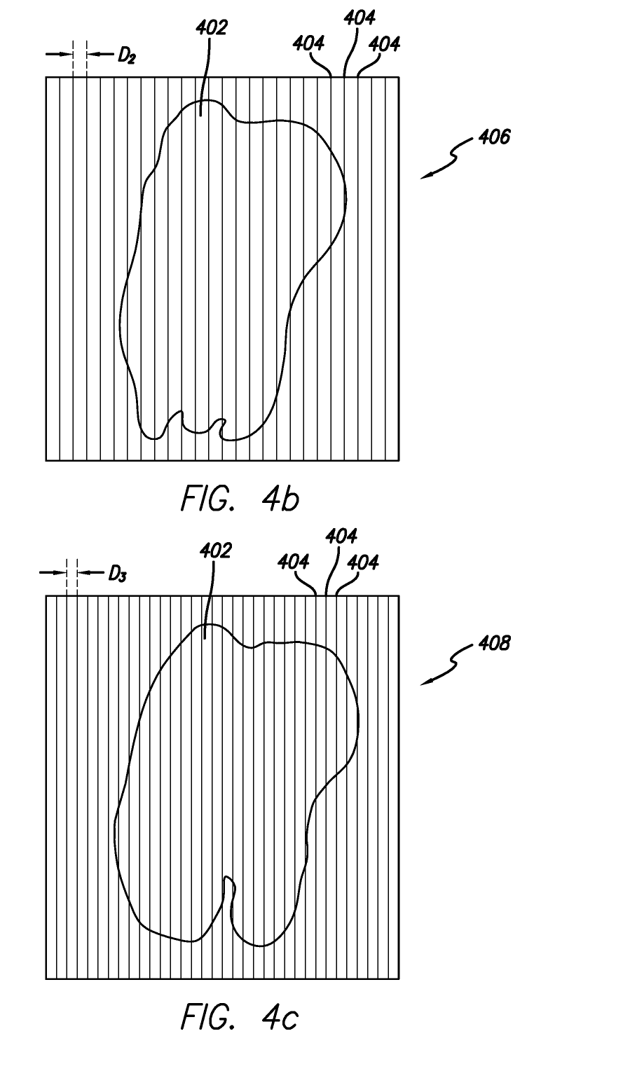

Regardless of the location of the array, the sensor is capable of generating electrical signals corresponding to a two-dimensional x-ray image of an object, such as a tooth, with an image of the array superimposed on the tooth. Using two or more such two-dimensional images of the same tooth, but with x-rays directed from different perspectives, sufficient data is generated from which a three-dimensional image can be derived using a CAD program or other algorithm. FIG. 4a is a simplified illustration of a first x-ray image 400 of a tooth 402 generated in accordance with an embodiment of the invention. The image 400 is taken by use of x-rays striking a sensor, such as the sensor 102 of FIG. 2, at a first average angle of incidence to the sensor. Because the sensor includes an array having elements adapted to absorb x-rays (or absorb light, depending upon the location of the array), the resulting x-ray image includes an image of the array elements 404 which are generally linearly-shaped and spaced apart from each other by a first distance D₁ and which are superimposed on the tooth 402.

FIG. 4b is a simplified illustration of a second x-ray image 406 of the same tooth 402 of FIG. 4a. This second image 406 is taken by adjusting the position of x-ray source relative to the sensor and the tooth, so that the x-rays strike the same sensor and tooth, but at a second average angle of incidence to the sensor that is different than the first average angle of incidence used for FIG. 4a. Thus the second x-ray image 406 includes the array elements superimposed on the tooth 402, but at a second distance D₂ between each array element that is different, and in this case less, than the first distance D₁ between each element in the first image 400. This second distance D₂ is less than the first distance D₁ as a result of the different average angle of incidence of the x-rays.

Similarly, FIG. 4c is a simplified illustration of a third x-ray image 408 of the same tooth 402 of FIGS. 4a and 4b. This third image 408 is taken by again adjusting the position of x-ray source relative to the sensor and the tooth, so that the x-rays strike the same sensor and tooth, but at a third average angle of incidence to the sensor that is different than the first and second average angles of incidence used for FIGS. 4a and 4b. Thus the third x-ray image 408 includes the array elements superimposed on the tooth 402, but at a third distance D₃ between each array element that is different, and in this case less, than the first and second distances D₁ and D₂, which again is due to yet another average angle of incidence of the x-rays.

Because the distance between each physical element in the array that is part of the sensor is fixed and is a known or predetermined physical distance, CAD programs or other algorithms can be used to generate a three-dimensional image of the tooth 402 based upon data corresponding to the predetermined physical distance and upon the image distances D₁, D₂ and D₃ corresponding to the imaged arrays that are superimposed upon the tooth 402 in the three images 400, 406, 408. In an alternative embodiment, as few as two different images may be all that is necessary to generate a three-dimensional image. However, the more images taken involving different x-ray angles of incidence, a three-dimensional image can be generated that is more accurate and detailed.

In summary, due to the change in angulations, i.e., the change in average angles of incidence of x-rays striking a sensor, the distances between the grid lines as shown in the images are altered for each image. This difference is then taken into consideration for the mathematically-derived, three-dimensional image of the object. Any subsequent images taken would contribute even more information and add accuracy to the rendered model.

Embodiments of the invention can now accurately generate a three-dimensional model with the appropriate CAD/CAM software or other software. Since the the thickness of each array element and the distance between each array element is known, the system can mathematically stitch the images together and render an accurate, three-dimensional model.

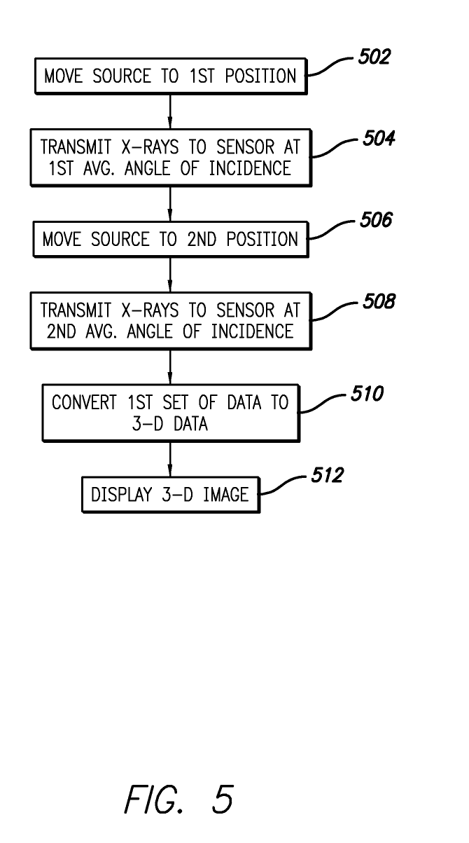

FIG. 5 is a flow diagram showing a method of obtaining a three-dimensional image of an object according to an embodiment of the invention. First, an x-ray source is moved into a first position in relation to an intraoral sensor so that a tooth is disposed between the intraoral sensor and the x-ray source. (Step 502) A first plurality of x-rays is transmitted from the x-ray source to the sensor at a first average angle of incidence to the sensor to produce a first set of electrical signals corresponding to a first two-dimensional image. (Step 504)

The x-ray source is moved to a second position in relation to the intraoral sensor and the tooth so that the tooth remains disposed between the sensor and the x-ray source. (Step 506) A second plurality of x-rays is transmitted from the x-ray source to the sensor at a second average angle of incidence to the sensor to produce a second set of electrical signals corresponding to a second two-dimensional image. (Step 508) A first set of data (corresponding to the first and second sets of electrical signals) is converted into a second set of data corresponding to a three-dimensional image. (Step 510) The second set of data is then used for presenting the three-dimensional image for display on a display screen. (Step 512).

Thus disclosed is an x-ray image sensor having an array disposed between an x-ray source and a photo-responsive device, such as a CCD or CMOS device. The array is comprised of a plurality of generally linearly-shaped, opaque elements that are spaced apart from each other by a predetermined distance and are adapted to absorb either x-ray radiation or light radiation. When a plurality of x-ray images is taken of the same object from different angles, images of the array are superimposed on the images of the object. Using a CAD program or other algorithm, the differences in spacing between the array elements for each image are used to derive a three-dimensional image of the object.

While the description above refers to particular embodiments of the present invention, it will be understood that many modifications may be made without departing from the spirit thereof. The claims are intended to cover such modifications as would fall within the true scope and spirit of the present invention. The presently disclosed embodiments are therefore to be considered in all respects as illustrative and not restrictive, the scope of the invention being indicated by the claims rather than the foregoing description, and all changes which come within the meaning and range of equivalency of the claims are therefore intended to be embraced therein.

What is claimed is:

1. An x-ray image sensor for obtaining a medical x-ray image by receiving x-rays originating from an x-ray source, comprising:
a housing;
a scintillator disposed in the housing and adapted to convert the x-rays to light signals;
a photo-responsive device disposed in the housing and adapted to convert the light signals to electric signals; and
an array disposed between the x-ray source and the photo-responsive device when the x-rays are being received, said array comprised of a plurality of generally linearly-shaped opaque elements, wherein each of the plurality of generally linearly-shaped opaque elements is adapted to absorb one of x-ray radiation and light radiation, and wherein at least a portion of the plurality of the generally linearly-shaped opaque elements are spaced apart from one another by a predetermined distance.

2. The sensor of claim 1 wherein the medical x-ray image is an image of a tooth and wherein the housing is adapted for insertion into a mouth of a person.

3. The sensor of claim 1 wherein the photo-responsive device is a charge coupled device.

4. The sensor of claim 1 wherein the photo-responsive device is a complementary metal oxide semiconductor device.

5. The sensor of claim 1 wherein the housing includes a housing wall having an outer surface and an inner surface, wherein the array is disposed one of on the outer surface, on the inner surface and between the outer and inner surfaces, and wherein each of the plurality of generally linearly-shaped opaque elements is adapted to absorb x-ray radiation.

6. The sensor of claim 1 wherein the scintillator has a proximate surface and a distal surface, wherein the proximate surface is adapted to receive the x-rays and the distal surface is adapted to emit the light signals when the scintillator converts the x-rays to light signals, wherein the array is disposed on the proximate surface, and wherein each of the plurality of generally linearly-shaped opaque elements is adapted to absorb x-ray radiation.

7. The sensor of claim 1 wherein the scintillator has a proximate surface and a distal surface, wherein the proximate surface is adapted to receive the x-rays and the distal surface is adapted to emit the light signals when the scintillator converts the x-rays to light signals, wherein the array is disposed on the distal surface, and wherein each of the plurality of generally linearly-shaped opaque elements is adapted to absorb light radiation.

8. The sensor of claim 1 wherein the scintillator and the photo-responsive device are disposed in a spaced-apart relationship, wherein the housing defines a cavity disposed between the scintillator and the photo-responsive device, wherein the array is disposed in the cavity, and wherein each of the plurality of generally linearly-shaped opaque elements is adapted to absorb light radiation.

9. The sensor of claim 1 wherein the housing includes a housing wall and defines a cavity disposed between the housing wall and the scintillator, wherein the array is disposed in the cavity, and wherein each of the plurality of generally linearly-shaped opaque elements is adapted to absorb x-ray radiation.

10. The sensor of claim 1 further comprising an optic device disposed in the housing between the scintillator and the photo-responsive device and adapted to channel the light signals from the scintillator to the photo-responsive device.

11. The sensor of claim 10 wherein the optic device has a proximate side and a distal side, wherein the proximate side is adapted to receive the light signals and the distal side is adapted to emit the light signals when the optic device channels the light signals from the scintillator to the photo-responsive device, wherein the array is disposed one of on the proximate side and on the distal side, and wherein each of the plurality of generally linearly-shaped opaque elements is adapted to absorb light radiation.

12. An x-ray image sensor for obtaining a medical x-ray image by receiving x-rays originating from an x-ray source, comprising:
a housing;
a scintillator disposed in the housing and adapted to convert a first portion of the x-rays to light signals; and
a photo-responsive device disposed in the housing and adapted to convert a first portion of the light signals to electric signals,
wherein the photo-responsive device includes a first array adapted to prevent conversion of a second portion of the light signals to other electric signals, wherein the first array includes a plurality of generally linearly-shaped elements, and wherein each of the plurality of generally linearly-shaped elements is disposed in a generally parallel, spaced-apart relationship from one another by a predetermined distance.

13. The sensor of claim 12 wherein the medical x-ray image is an image of a tooth and wherein the housing is adapted for insertion into a mouth of a person.

14. The sensor of claim 12 further comprising an optic device disposed in the housing between the scintillator and the photo-responsive device and adapted to channel the light signals from the scintillator to the photo-responsive device.

15. The sensor of claim 12 wherein the photo-responsive device is a CCD having a capacitor array, wherein the plurality of generally linearly-shaped elements is comprised of one of a plurality of disabled capacitors and a plurality of omitted capacitors disposed within the capacitor array.

16. The sensor of claim 12 wherein the photo-responsive device is a CMOS device having a pixel array, wherein the plurality of generally linearly-shaped elements is comprised of one of a plurality of disabled pixels and a plurality of omitted pixels disposed within the pixel array.

17. The sensor of claim 12 wherein the photo-responsive device is a charge coupled device having a capacitor array, wherein the first array is comprised of a mask disposed on the capacitor array, and wherein each of the plurality of generally linearly-shaped elements is adapted to absorb light radiation.

18. The sensor of claim 12 wherein the photo-responsive device is a complementary metal oxide semiconductor device having a pixel array, wherein the first array is comprised of a mask disposed on the pixel array, and wherein each of the plurality of generally linearly-shaped elements is adapted to absorb light radiation.

19. An apparatus for providing a three-dimensional dental x-ray image using x-rays originating from an x-ray source, the apparatus comprising:
an intraoral sensor adapted to produce electrical signals corresponding to a plurality of two-dimensional dental x-ray images; and
a computer system adapted to receive the electrical signals from the intraoral sensor, wherein the computer system includes a program and a processor adapted to execute the program, wherein the program is adapted to convert a first set of data corresponding to the electrical signals into a second set of data corresponding to the three-dimensional dental x-ray image, wherein the intraoral sensor includes:
a housing;
a scintillator disposed in the housing and adapted to convert the x-rays to light signals;
a photo-responsive device disposed in the housing and adapted to convert the light signals to the electric signals; and
an array disposed between the x-ray source and the photo-responsive device when the intraoral sensor is producing the electrical signals, said array comprised of a plurality of generally linearly-shaped opaque elements, wherein each of the plurality of generally linearly-shaped opaque elements is adapted to absorb one of x-ray radiation and light radiation, and wherein at least a portion of the plurality of the generally linearly-shaped opaque elements are spaced apart from one another by a predetermined distance.

20. The apparatus of claim 19 wherein the photo-responsive device is a charge coupled device.

21. The apparatus of claim 19 wherein the photo-responsive device is a complementary metal oxide semiconductor device.

22. The apparatus of claim 19 wherein the housing includes a housing wall having an outer surface and an inner surface, wherein the array is disposed one of on the outer surface, on the inner surface and between the outer and inner surfaces, and wherein each of the plurality of generally linearly-shaped opaque elements is adapted to absorb x-ray radiation.

23. A method of obtaining a dental image, comprising:
moving an x-ray source into a first position in relation to an intraoral sensor and a tooth so that the tooth is disposed between the intraoral sensor and the x-ray source;
transmitting a first plurality of x-rays from the x-ray source to the intraoral sensor to produce a first set of electrical signals corresponding to a first two-dimensional image, wherein the first plurality of x-rays are transmitted at a first average angle of incidence to the intraoral sensor;
moving the x-ray source into a second position in relation to the intraoral sensor and the tooth so that the tooth is disposed between the intraoral sensor and the x-ray source after transmitting the first plurality of x-rays;
transmitting a second plurality of x-rays from the x-ray source to the intraoral sensor to produce a second set of electrical signals corresponding to a second two-dimensional image, wherein the second plurality of x-rays are transmitted at a second average angle of incidence to the intraoral sensor, and wherein the second average angle of incidence is different than the first average angle of incidence; and
converting a first set of data into a second set of data, wherein the first set of data corresponds to the first and second sets of electrical signals, and wherein the second set of data corresponds to a three-dimensional image,
wherein the intraoral sensor includes an array comprised of a plurality of generally linearly-shaped opaque elements, wherein each of the plurality of generally linearly-shaped opaque elements is adapted to absorb one of x-ray radiation and light radiation, and wherein at least a portion of the plurality of the generally linearly-shaped opaque elements are spaced apart from one another by a predetermined distance.

24. The method of claim 23 wherein the intraoral sensor further includes a charge coupled device.

25. The method of claim 23 wherein the intraoral sensor further includes a complementary metal oxide semiconductor device.

26. The method of claim 23 wherein the intraoral sensor further includes a housing having a housing wall with an outer surface and an inner surface, wherein the array is disposed one of on the outer surface, on the inner surface and between the outer and inner surfaces, and wherein each of the plurality of generally linearly-shaped opaque elements is adapted to absorb x-ray radiation.

27. An apparatus for providing a three-dimensional dental x-ray image using x-rays originating from an x-ray source, the apparatus comprising:
means for producing a first set of electrical signals from a first plurality of x-rays and a second set of electrical signals from a second plurality of x-rays, wherein the first set of electrical signals corresponds to a first two-dimensional image and the second set of electrical signals corresponds to a second two-dimensional image; and
means for converting a first set of data into a second set of data, wherein the first set of data corresponds to the first and second sets of electrical signals, and wherein the second set of data corresponds to the three-dimensional dental x-ray image,
wherein the means for producing the first and second sets of electrical signals includes an array comprised of a plurality of generally linearly-shaped opaque elements, wherein each of the plurality of generally linearly-shaped opaque elements is adapted to absorb one of x-ray radiation and light radiation, and wherein at least a portion of the plurality of the generally linearly-shaped opaque elements are spaced apart from one another by a predetermined distance.
